SEQUENCE LISTING (10 sequences)

SEQ ID NO: 1 - protein, 309 aa, synthetic construct
MIFLAILDLKSLVLNAINYWGPKNNNGIQGGDFGYPISEKQIDTSIITSTHPRLIPHDLTIPQNLETIFTTTQVLTNNTDLQQSQTVSFAKKTTTTTSTSTTNGWTEGGKISDTLEEKVSVSIPFIGEGGGKNSTTIEANFAHNSSTTTFQQASTDIEWNISQPVLVPPRKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLRTIEKTWYARHATLHNGKKISINNVTEMAPTSPIKTN

SEQ ID NO: 2 - protein, 309 aa, synthetic construct
MIFLAILDLKSLVLNAINYWGPKNNNGIQGGDFGYPISEAQIDTSIITSTHPRLIPHDLTIPQNLETIFTTTQVLTNNTDLQQSQTVSFAKKTTTTTSTSTTNGWTEGGAISDTLEEKVSVSIPFIGEGGGKNSTTIEANFAHNSSTTTFQQASTDIEWNISQPVLVPPAKQVVATLVIMGGNFTIPMDLMTTIDSTEHYSGYPILTWISSPDNSYNGPFMSWYFANWPNLPSGFGPLNSDNTVTYTGSVVSQVSAGVYATVRFDQYDIHNLATIEKTWYARHATLHNGKKISINNVTEMAPTSPIKTN

SEQ ID NO: 3 - DNA, 930 bp, synthetic construct
  source 1..930: mol_type = other DNA
atgatttttttggctatactagatttaaaatcactggtgttgaatgcgatcaactattggggtccgaaaaacaacaacggtattcagggtggtgattttggttacccgattagcgaaaagcagattgacaccagcattattacctctacccacccgcgtctgattccgcatgatcttacgatcccgcagaatttggaaaccatttttaccactacgcaagtcctgacgaacaacaccgacctgcagcaaagccaaaccgttagctttgctaaaaagaccactaccacgacctcgacctctacgacgaacggctggaccgagggcggcaagatctccgataccttggaggaaaaggtgtccgtgtctatcccgttcatcggcgaaggcggcggtaagaactccaccacaatcgaggcgaatttcgcgcataactcctcaacgaccacatttcaacaggcaagcaccgacatcgagtggaatatttcccaaccggttctggtgccgccgcgcaaacaggttgttgcgaccttggttattatgggtggcaatttcaccatcccgatggatttaatgaccacgatcgacagcactgagcactacagcggttacccgatcctgacctggattagcagcccggataactcctacaatggcccattcatgagctggtacttcgcgaactggccgaatctgccgagcggtttcggcccactgaacagcgacaacacggttacctataccggtagcgttgtctcgcaagtgagcgcgggggtgtatgccacggtgcgttttgatcagtatgacatccacaatctgcgcaccatcgagaagacctggtacgcccgtcatgctaccctgcacaacggtaaaaaaatcagcatcaataacgtgaccgaaatggcaccgacctctccgatcaaaaccaactaa

SEQ ID NO: 4 - DNA, 930 bp, synthetic construct
  source 1..930: mol_type = other DNA
atgatttttttggctatactagatttaaaatccctggttttgaatgcgatcaactactggggtccgaagaacaacaacggcatccaaggtggtgatttcggttatccgatcagcgaagcccagattgacacctcaatcatcacctccacccatccgcgcctgattccgcacgatttgaccatcccgcaaaacttggaaaccatctttaccacgacccaggtactgaccaacaacaccgacctgcaacagagccaaaccgtgtcttttgctaaaaagactaccacgacgacctctacctctacaacgaacggctggaccgagggtggcgcaatctccgacaccctggaggaaaaggtgagcgtttctatcccgttcattggtgaaggcggcggaaaaaacagcactacgattgaagcgaacttcgcgcacaacagcagcactacgacctttcagcaagcgagcaccgatattgagtggaatatttcccagccggttctggtgccgcctgcaaagcaggtcgtggccactctggttattatgggtggtaatttcaccataccgatggatttaatgaccacgattgatagcaccgagcactacagcggctacccgatcctgacctggattagcagcccggataatagttataatggtccgttcatgagctggtacttcgccaactggccaaatcttccgagcggctttggcccactgaattctgacaacaccgtgacctataccggtagcgtggtctcgcaagtgagcgcgggtgtttatgcgaccgttcgttttgaccagtacgacatccacaatctcgcgaccatcgagaaaacctggtacgctcgtcatgctaccctgcataacggcaaaaagatcagcattaataacgttacggagatggcaccgacctcaccgattaaaaccaactaa

SEQ ID NO: 5 - DNA, 1992 bp, Zea mays
  source 1..1992: mol_type = genomic DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 6 - DNA, 253 bp, Agrobacterium tumefaciens
  source 1..253: mol_type = genomic DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 7 - DNA, 1026 bp, Salmonella enterica
  source 1..1026: mol_type = genomic DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 8 - DNA, 20 bp, synthetic construct
  source 1..20: mol_type = other DNA
cagggtgtcacgttgcaaga

SEQ ID NO: 9 - DNA, 20 bp, synthetic construct
  source 1..20: mol_type = other DNA
ccgctcgtctggctaagatc

SEQ ID NO: 10 - DNA, 24 bp, synthetic construct
  source 1..24: mol_type = other DNA
tgcctgaaaccgaactgcccgctg

(19) United States
(12) Patent Application Publication
(10) Pub. No.: US 2023/0257766 A1
(43) Pub. Date: Aug. 17, 2023

(54) Use of insecticidal protein

(71) Applicant: BEIJING DABEINONG BIOTECHNOLOGY CO., LTD., Beijing (CN)

(72) Inventors: Tengyu CHANG, Beijing (CN); Qinyang WANG, Beijing (CN); Aihong ZHANG, Beijing (CN); Qing TAO, Beijing (CN)

(21) Appl. No.: 18/062,593

(22) Filed: Dec. 7, 2022

(30) Foreign Application Priority Data
Dec. 13, 2021 (CN) 202111516734.1

(51) Int. Cl. C12N 15/82; A01N 63/50

(52) U.S. Cl. CPC C12N 15/8286; A01N 63/50

(57) ABSTRACT

Related is a use of an insecticidal protein. The insecticidal protein may be used to control a thrip pest. A method for controlling the a thrip pest includes: allowing the a thrip pest to be at least in contact with an ACh1 protein. In the present application, the a thrip pest is controlled through producing the ACh1 protein that can kill the a thrip pest in bacteria and/or a plant in vivo.

### CROSS-REFERENCE TO RELATED APPLICATION

The present application is based on and claims priority to Chinese application with a CN application number of 202111516734.1 and an application date of Dec. 13, 2021, the disclosure of which is hereby incorporated by reference again in its entirety.

### SEQUENCE LISTING

The instant application contains a Sequence Listing which has submitted electronically in ASCII format and is hereby incorporated by reference in its entirety. Said ASCII copy is named PN 192849 SEQ LIST.xml and is 15,341 bytes in size. The sequence listing contains 10 sequences, which is identical in substance to the sequences disclosed in the CN application except that the priority is added and includes no new matter.

### TECHNICAL FIELD

The present disclosure relates to a use of an insecticidal protein, and in particular, to a use of an ACh1 protein for controlling damage of a thrip pest to a plant by expressing in the plant.

### BACKGROUND

Adults of Thysanoptera all have two pairs of fringed-wings, i.e., wings with tassels like red tassels on the edges thereof, and thus such type of insects are classified as “Thysanoptera”. Moreover, many species of Thysanoptera insects like to move in flowers of a kind of Compositae plant-thistle, such as Cirsium japonicum and Cirsium setosum, and thus they are also called “thrips”.

Thrips, as a general name of Thysanoptera insects, are important economic pests. For example, Anaphothrips obscures does harm to maize, wheat, barley and the like gramineous crops, resulting in intermittent silvery white stripes on the back of leaves accompanied by small stains and yellow stripes on the part on the front of the leaves opposite to the silvery white stripes, and resulting in yellow withered leaves and even destruction of species as caused by serious damages.

Frankliniella occidentalis (Pergande), also known as alfalfa thrip, is omnivorous. It originated in Americas and invaded China, and now has been found all over China. Frankliniella occidentalis (Pergande) does harm to maize, cotton, soybean, cucumber, tomato and the like crops, causing petals to fade, leaves to shrink, and scars to be formed on stems and fruits, which may eventually make a plant wither, and at the same time spread many viruses including a tomato spotted wilt virus. Frankliniella occidentalis (Pergande) has very strong reproductive capacity, tiny individuals and great concealment, so it is difficult to effectively control it in the field. At a stable temperature in a greenhouse, 12-15 generations of it can occur continuously in a year, and female insects conduct bisexual reproduction and parthenogenesis. It can develop at 15° C.-35° C., and it only takes 14 days from an egg to an adult. The most eggs are laid at 27.2° C., and one female can lay 229 eggs. On a usual host plant, it develops rapidly and has a very strong reproductive capacity.

Corn is an important food crop in China. The a thrip pest causes huge food losses each year, and even affects the living conditions of the local population. In order to control the a thrip pest, main control methods usually used by people are agricultural control, chemical control, physical control and biological control.

The agricultural control is to comprehensively coordinate and manage an entire farmland ecosystem from multi-factors, and regulate crops, pests, and environmental factors so as to create a farmland ecological environment that facilitates crop growth but not facilitates the occurrence of the a thrip pest. For example, it is achieved by strengthening water and fertilizer management, promoting healthy and strong growth of a plant, and reducing harm. However, this manner needs more labor and is not suitable for the current trend of agricultural industrialization.

The chemical control is pesticide control, which uses chemical pesticides to kill pests and is an important part of the comprehensive management of the a thrip pest. The chemical control has the characteristics of rapidity, convenience, simplicity and high economic benefits, and is an essential emergency measure, especially in the case of a large occurrence of the chemical control. At present, a chemical control method is mainly using a conventional agent such as imidacloprid, acetamiprid and the like to spray. However, due to the short reproduction period and large reproduction quantity of the thrips, drug resistance is produced quickly. Moreover, frankliniella occidentalis (Pergande) does harm to flower organs, often hides in the axils of stamens and petals, and thus it is difficult to contact them even upon application of an agent, which also leads to the low control effect of a contact insecticide on frankliniella occidentalis.

Physical control is mainly based on the response of pests to various physical factors in environmental conditions, using various physical factors such as light, electricity, color, temperature and humidity, as well as mechanical devices for trapping, radiation sterility and other methods to control pests. Most of the thrips are liable to be attracted by yellow, so a yellow sticky board can be hanging to reduce the harm of the thrips when planting is conducted in a greenhouse, but this method actually has little effect in the field.

The biological control is the use of some beneficial organisms or biological metabolites to control the number of pest populations in order to achieve a purpose of reducing or eliminating the pests, such as utilizing parasitic natural enemies, predatory natural enemies and pathogenic natural enemies, etc. to suppress the population size of the pest or eliminate the pest. It is characterized by safety to people and livestock, less environmental pollution, and long-term control of some pests. For the thrips, there are predatory stinkbugs, predatory mites, parasitic wasps and parasitic fungi, etc. among which the predatory natural enemies are the most effective. However, no matter what kind of natural enemies, they all need a suitable environment for colonization and reproduction. However, the current farmland ecosystem is not suitable for the colonization of a large number of natural enemies, which leads to repeated application of the biological control and thus increase of the use cost, while the control effect is still not ideal.

In order to solve the limitations of the agricultural control, the chemical control, physical control and the biological control in practical applications, scientists found that some insect-resistant transgenic plants may be obtained by transferring insect-resistant genes encoding insecticidal proteins into plants so as to control plant pests.

Pest-resistant crops have been developed by genetically engineering crops to introduce a Bacillus thuringiensis (Bt) protein into crops. For example, Cry1Ab is used to develop corns resistant to corn borer. At present, these genetically modified crops are widely used in agriculture and provide farmers with an environmentally friendly alternative to traditional insect control methods. Although the genetically modified crops have been shown to be quite effective against lepidopteran pests (the corn borer, bollworm, and the like), no genetically modified crops have been found that can control the a thrip pest. The main reason for this is that no Cry protein has been found to be virulent to the a thrip pest.

ACh1 is a new class of insecticidal proteins, which is completely different from the traditional Bt protein. By analyzing a protein secondary structure, the protein is speculated to belong to a β- pore forming protein. The mechanism of action of such proteins is generally enzymatic cleavage activation, binding with receptors, formation of oligomers, and pore-forming on membrane surfaces. The enzymatic cleavage activation in insect gut, receptor binding on the insect gut and a physicochemical environment in the insect gut determine whether the transmembrane pore can form in cell membranes of the insect gut. After such type of protein is secreted by the bacteria, it needs to be digested in a target body to form an active protein. The enzyme cleavage process is mainly performed at an amino-terminal or carboxyl-terminal of the protein, to turn the protein into an active fragment. The active fragment binds to a receptor on an epithelial cell membrane of the insect gut to form oligomer, and inserts into an gut membrane, so that a transmembrane pore appears on the cell membrane, and the osmotic pressure change and pH balance and the like inside and outside the cell membrane are destroyed, and the digestion process of the insects is disrupted, finally resulting in death of the insects.

The ACh1 protein has been reported to have inhibitory activity against silkworm and potato beetles. However, there is no report on the control of plant damage by the a thrip pest by producing transgenic plants expressing the ACh1_1 and the ACh1_4 protein so far.

### SUMMARY

The present disclosure is intended to provide a use of an insecticidal protein, and for the first time provide a method for controlling a thrip pest by producing a transgenic plant expressing an ACh1 protein, to effectively overcome technical defects in agricultural control, chemical control, physical control and biological control in the prior arts.

In order to achieve the above objective, the present disclosure provides a method for controlling a thrip pest, including allowing the Monolepta hieroglyphica to be at least in contact with an ACh1 protein.

Preferably, the ACh1 protein is present in a host cell that produces at least the ACh1 protein, and the a thrip pest is in contact with at least the ACh1 protein by ingesting the host cell.

More preferably, the ACh1 protein is present in a bacterium or a transgenic plant that produces at least the ACh1 protein. The a thrip pest is in contact with at least the ACh1 protein by ingesting the bacterium or a tissue of the transgenic plant. After contacting, the growth of the a thrip pest is inhibited and/or death is caused, so as to achieve the control of the damage of the a thrip pest to plants.

The transgenic plant may be in any growth stages.

The tissue of the transgenic plant is a fruit, a male ear, a female ear, an anther, or a filament.

The control of the damage of the a thrip pest to the plants does not vary with the planting location and/or the planting time.

The plant is corn, soybean, cotton or rape.

A step before the contacting step is to plant a plant containing a polynucleotide encoding the ACh1 protein.

On the basis of the above technical solution, the ACh1 protein is an ACh1_1 protein or an ACh1_4 protein.

Preferably, the ACh1 protein has an amino acid sequence shown in SEQ ID NO:1 or SEQ ID NO:2.

On the basis of the above technical solution, the plant further includes at least a second nucleotide different from the nucleotide encoding the ACh1 protein.

Further, the second nucleotide encodes a Cry-like insecticidal protein, a Vip-like insecticidal protein, a protease inhibitor, lectin, α-amylase, or a peroxidase.

Optionally, the second nucleotide is a dsRNA that inhibits an important gene in a target insect pest.

The thrip pest is selected from the group consisting of Anaphothrips obscurus, Frankliniella tenuicornis (Uzel), Stenchaetothrips biformis (Bagnall) and frankliniella occidentalis (Pergande).

In order to achieve the above objective, the present disclosure further provides a use of an ACh1 protein for controlling a thrip pest.

In order to achieve the above objective, the present disclosure further provides a method for producing a plant for controlling a thrip pest, including introducing a polynucleotide sequence encoding an ACh1 protein into a genome of the plant.

In order to achieve the above objective, the present disclosure further provides a method for producing a plant seed for controlling a thrip pest, including hybridizing a first plant obtained by the method with a second plant, so as to produce a seed containing a polynucleotide sequence encoding an ACh1 protein.

In order to achieve the above objective, the present disclosure further provides a method for cultivating a plant for controlling a thrip pest, including: at least one plant seed is planted, and the genome of the plant seed includes a polynucleotide sequence encoding an ACh1 protein; the plant seed is grown into a plant, and the plant is grown under conditions that the a thrip pest is artificially inoculated and/or the hazard of the a thrip pest naturally occurs, and a plant that has an attenuated plant damage and/or has an increased plant yield compared with other plants that do not have the polynucleotide sequences encoding the ACh1 protein is harvested.

The “contact” in the present disclosure means that insects and/or pests touch, stay and/or feed on a plant, a plant organ, a plant tissue or a plant cell, and the plant, plant organ, plant tissue or plant cell may be to express the insecticidal protein in vivo, or the plant, plant organ, plant tissue or plant cell has the insecticidal protein on the surface and/or has a microorganism that produces the insecticidal protein.

A term “control” and/or “prevention” in the present disclosure means that the a thrip pest is in contact with at least the ACh1 protein, and the growth of the a thrip pest is inhibited and/or death is caused after the contact. Further, the a thrip pest is in contact with at least the ACh1 protein by ingesting the plant tissue, and after the contact, all or part of the a thrip pest is inhibited in growth and/or death is caused. The inhibition refers to sub-lethal, namely it is not lethal but may cause a certain effect in growth, behavior, physiology, biochemistry and tissue and other aspects, such as slow growth and/or stop. At the same time, the plant should be morphologically normal, and may be cultivated by a conventional method for consumption and/or generation of products. In addition, the plant and/or plant seed containing the polynucleotide sequence encoding the ACh1 protein for controlling the a thrip pest, under the condition that the a thrip pest is artificially inoculated and/or the a thrip pest naturally occurs, has the reduced plant damage compared with non-transgenic wild-type plants, and the specific manifestations include, but are not limited to, improved stem resistance, and/or increased grain weight, and/or increased yield, and the like. The “control” and/or “prevention” effect of the ACh1 protein on the a thrip pest may exist independently, and may not be weakened and/or disappeared due to the presence of other substances that may “control” and/or “prevent” the a thrip pest. Specifically, if any tissue of the transgenic plant (containing the polynucleotide sequence encoding the ACh1 protein) simultaneously and/or asynchronously exist with and/or produce the ACh1 protein and/or another substance that may control the a thrip pest, the existence of the another substance neither affects the “control” and/or “prevention” effect of the ACh1 protein on the a thrip pest, nor may cause the “control” and/or “prevention” effect to be completely and/or partially implemented by the another substance, which is independent of the ACh1 protein. Usually, in the field, the ingestion process of the plant tissue by the a thrip pest is short and difficult to observe with naked eyes. Therefore, under the condition that the a thrip pest is artificially inoculated and/or the a thrip pest naturally occurs, for example, any tissues of the transgenic plant (containing the polynucleotide sequence encoding the ACh1 protein) have the dead a thrip pest, and/or the a thrip pest on which the growth is inhibited, and/or have the reduced plant damage compared with the non-transgenic wild-type plants, the method and/or the use of the present disclosure is achieved. That is to say, the method and/or the use for controlling the a thrip pest is achieved by allowing the thrip pest to be at least in contact with the ACh1 protein.

In the present disclosure, the expression of the ACh1 protein in a transgenic plant may be accompanied by the expression of one or more Cry-like insecticidal proteins and/or Vip-like insecticidal proteins. Co-expression of such more than one insecticidal toxin in the same transgenic plant may be achieved by genetically engineering the plant to contain and express a desired gene. In addition, one plant (first parent) may express the ACh1 protein by a genetic engineering operation, and a second plant (second parent) may express the Cry-like insecticidal proteins and/or Vip-like insecticidal proteins by the genetic engineering operation. Offspring plants expressing all the genes introduced into the first and second parents are obtained by hybridizing the first and second parents.

RNA interference (RNAi) refers to a phenomenon that is highly conserved during the evolution process and induced by a double-stranded RNA (dsRNA), and a homologous mRNA is efficiently and specifically degraded. Therefore, an RNAi technology may be used in the present disclosure to specifically knock out or shut down the expression of a specific gene in the target insect pest.

The frankliniella occidentalis (Pergande) described in the present application belongs to the genus Frankliniella of Thripidae in Thysanoptera. The egg of it is kidney-shaped, white, and 0.25 mm long, and the egg stage is 5-15 days in the field, while the average egg stage is 2.6 days at 25° C. Nymph: it has 2 instars. The nymph begins to feed immediately after hatching. The newly hatched nymph has a white body, and turns yellow before molting. The 2nd instar nymph is waxy yellow, very active, and has food intake which is 3 times that of the 1st instar nymph. When close to maturity, it shows negative phototaxis, leaving the plant and entering the soil. The developmental threshold temperature of the nymph is 9.4° C., and the development duration of the nymph in the field is 9-12 days, which can be extended to 60 days in winter, while the development of the 1st and 2nd instar nymphs only takes 2.3 and 3.7 days under a condition of a constant temperature of 25° C. The nymphs and the adults often feed in small groups. The adults are tiny, with an average body length of 1.5 mm. The wings of it are narrow, and the tassels at the leading edge of the wings are significantly shorter than those at the trailing edge. It can fly and jump well, and can make short-distance migration with the help of airflow. The body color of it ranges from light yellow to brown, and the antennae of it has 8 segments. Female insects often lay their eggs in mesophyll tissues, inflorescences or young fruits.

The frankliniella occidentalis (Pergande) is omnivorous, and has more than 500 known host plants, including important crops such as Compositae, Cucurbitaceae, Leguminosae, Cruciferae, etc., mainly including plum, peach, apple, grape, strawberry, eggplant, pepper, lettuce, tomato, bean, orchid, chrysanthemum, etc. With the continuous diffusion and extension of frankliniella occidentalis (Pergande), its host species have been continuously increasing. For different kinds of host plants, frankliniella occidentalis (Pergande) has different preference degrees, but they all can survive and have considerable reproductive capacity. Frankliniella occidentalis (Pergande) pierces and sucks the juice of leaves, buds, flowers or solanberries of a host plant with a special mouthpart. The damaged leaves are presented with white spots at first and then become patches. The front of the leaves looks like suffering from a spot disease, and the back of the leaves has black feculae. When the host plant is seriously damaged, the leaves become smaller and shrivelled, or even the flowers are yellowed, withered and wilted. The damaged floral organs are presented with white spots or brown, and scratches or even scars are often leaved on the damaged fruits. After damaged, the flower crops are presented with faded leaves and petals and eating scars left thereon, which affects the appearance and commercial value of the flowers. The infected buds and flowers are deformed, and in severe cases, the flowers cannot bloom normally. The long-distance diffusion of the frankliniella occidentalis (Pergande) mainly depends on human factors. The allocation and transportation of seedlings, flowers and other agricultural products, especially the transportation and artificial carrying of cut flowers, is the main way of its long-distance transmission. The frankliniella occidentalis (Pergande) has strong viability, and can still survive after the products are transported and sold to other cities. Moreover, this pest is easy to be blown away with the wind, and it is easy to be carried and spread with clothes, transportation tools, etc. The frankliniella occidentalis (Pergande) is easy to transmit virus diseases, and it has been listed as a quarantine object in China.

The ACh1 protein belongs to a class of β- pore-forming proteins, and the enzymatic cleavage activation in insect gut, receptor binding on the insect gut and a physicochemical environment in the insect gut are key points for achieving the effect of a β- pore-forming protein. Only after the β-pore forming protein can be digested into active fragments and bound to the receptor on an epithelial cell membrane of the insect gut, it is possible to make a certain β-pore forming protein have an inhibitory activity aganist the pests. The receptor binding process requires accurate matching, and often a single amino acid difference in the pore forming protein or receptor protein can cause changes in binding to the same receptor. For example, after an aerolysin protein belonging to the same β-pore forming protein has qualitative changes in the virulence of a CTLL-2 cell line after R336A mutation (Osusky, Teschk et al, 2008). Likewise, since the receptor is changed, the virulence of the same β-pore forming protein may also be changed. For example, dsRNA is used to inhibit a HAVCR1 gene in a MDCK cell line, resulting in a hundred-fold difference in the virulence of an epsilon-toxin protein on cells (Ivie, Fennessey et al, 2011). The above fully indicates that the interaction between the β-pore forming protein and enzymes and receptors in insects is complex and unpredictable.

The genome of the plant, plant tissue or plant cell in the present disclosure refers to any genetic materials in the plant, plant tissue or plant cell, and includes a cell nucleus and plastid and mitochondrial genome.

The polynucleotide and/or nucleotide described in the present disclosure forms a complete “gene” that encodes a protein or polypeptide in the required host cell. It is very easily recognized by those skilled in the art that the polynucleotide and/or nucleotide of the present disclosure may be placed under the control of a regulatory sequence in the target host.

It is well-known to those skilled in the art that DNA typically exists in a double-stranded form. In this arrangement, one strand is complementary to the other strand and vice versa. Other complementary strands of DNA are produced as a result of DNA replication in the plants. In this way, the present disclosure includes the use of the polynucleotide exemplified in a sequence listing and complementary strands thereof. A “coding strand” as commonly used in the field refers to a strand that binds to an antisense strand. In order to express a protein in vivo, typically one strand of DNA is transcribed into a complementary strand of mRNA, it serves as a template for translation of the protein. mRNA is actually transcribed from the “antisense” strand of DNA. The “sense” or “coding” strand has a series of codons (the codon is three nucleotides, and a specific amino acid may be produced by reading three at a time), and it may be read as an open reading frame (ORF) to form a target protein or peptide. The present disclosure also includes RNA that is functionally equivalent to the exemplified DNA.

The nucleic acid molecule or fragment thereof in the present disclosure hybridizes to the ACh1 gene of the present disclosure under stringent conditions. Any conventional nucleic acid hybridization or amplification methods may be used to identify the presence of the ACh1 gene of the present disclosure. The nucleic acid molecule or fragment thereof is capable of specifically hybridizing with other nucleic acid molecules under certain circumstances. In the present disclosure, if two nucleic acid molecules may form an anti-parallel double-stranded nucleic acid structure, it may be said that the two nucleic acid molecules may specifically hybridize with each other. If the two nucleic acid molecules show complete complementarity, one nucleic acid molecule is said to be a “complement” of the other nucleic acid molecule. In the present disclosure, while each nucleotide of one nucleic acid molecule is complementary to the corresponding nucleotide of the other nucleic acid molecule, the two nucleic acid molecules are said to show the “complete complementarity”. If the two nucleic acid molecules may hybridize to each other with sufficient stability such that they anneal and bind to each other under at least conventional “low stringency” conditions, the two nucleic acid molecules are said to be “minimally complementary”. Similarly, if the two nucleic acid molecules may hybridize to each other with the sufficient stability such that they anneal and bind to each other under conventional “high stringency” conditions, the two nucleic acid molecules are said to have “complementarity”. Deviation from the complete complementarity is permissible as long as such deviation does not completely prevent the two molecules from forming the double-stranded structure. In order for a nucleic acid molecule to function as a primer or a probe, it only needs to be sufficiently complementary in its sequence, as to allow for the formation of the stable double-stranded structure under adopted particular solvent and salt concentration.

In the present disclosure, the substantially homologous sequence is a section of a nucleic acid molecule, the nucleic acid molecule may specifically hybridize with a complementary strand of another matched nucleic acid molecule under highly stringent conditions. Suitable stringent conditions to promote the DNA hybridization, for example, treatment with 6.0x sodium chloride/sodium citrate (SSC) at about 45° C., and followed by washing with 2.0× SSC at 50° C., are well-known to those skilled in the art. For example, the salt concentration in a washing step may be selected from about 2.0×SSC and 50° C. under the low stringency conditions to about 0.2×SSC and 50° C. under the high stringency conditions. In addition, the temperature condition in the washing step may be increased from about 22° C. at a room temperature under the low stringency conditions to about 65° C. under the high stringency conditions. Both the temperature condition and the salt concentration may be changed, or one of which may be kept unchanged while the other variable is changed. Preferably, the stringency condition described in the present disclosure may be specific hybridization in 6×SSC and 0.5% sodium dodecyl sulfate (SDS) solutions at 65° C., and then membrane-washing once with 2×SSC, 0.1 % SDS and 1×SSC and 0.1 % SDS.

Therefore, sequences that have the insecticidal activity and hybridize to SEQ ID NO:3 or SEQ ID NO:4 of the present disclosure under the stringency condition are included in the present disclosure. These sequences have at least about 40%-50% of the identity with the sequences of the present disclosure, about 60%, 65% or 70% of the identity, even at least about 75%, 80%, 85%, 90%, 91%, 92%, 93%, 94%, 95%, 96%, 97%, 98%, 99% or greater sequence identity.

The genes and proteins described in the present disclosure include not only a specific exemplified sequence, but also include parts and/or fragments (including internal and/or terminal deletion as compared with a full-length protein) that preserve the characteristics of the insecticidal activity of the specific exemplified protein, a variant, a mutant, a substitute (a protein with a substituted amino acid), a chimera and a fusion protein. The “variant” or “variation” refers to a nucleotide sequence encoding the same protein or encoding an equivalent protein with the insecticidal activity. The “equivalent protein” refers to a protein that has the same or substantially the same biological activity against the a thrip pest as the claimed protein.

A “fragment” or “truncation” of the DNA molecule or protein sequence described in the present disclosure refers to a portion of the original DNA or protein sequence (nucleotide or amino acid) involved or an artificially modified form thereof (for example, a sequence suitable for plant expression), the length of the aforementioned sequence may have a change but is long enough to ensure that the (encoded) protein is an insect toxin.

A standard technology may be used to modify the gene and construct the genetic variant easily, for example, a technology for manufacturing a point mutation which is well-known in the field. As another example, U.S. Pat. No. 5605793 describes a method for producing additional molecular diversity using DNA reassembly after random fragmentation. The fragment of the full-length gene may be manufactured by using a commercial endonuclease, and an exonuclease may be used according to a standard procedure. For example, enzymes such as Bal31 or site-directed mutagenesis may be used to systematically excise nucleotides from the ends of these genes. The genes encoding the active fragments may also be obtained by using a plurality of restriction enzymes. The active fragments of these toxins may be obtained directly by using proteases.

The present disclosure may derive equivalent proteins and/or genes encoding these equivalent proteins from a β-pore forming protein isolate and/or a DNA library. There are various ways to obtain the insecticidal protein of the present disclosure. For example, antibodies of the insecticidal protein disclosed and claimed in the present disclosure may be used to identify and isolate other proteins from protein mixtures. In particular, the antibodies may arise from a portion of the protein that is most constant and most different from other β-pore forming proteins. These antibodies may then be used to specifically identify the equivalent proteins with the characteristic activity by immunoprecipitation, an enzyme-linked immunosorbent assay (ELISA), or a western blotting method. Antibodies of the proteins or the equivalent proteins or the fragments of such proteins disclosed in the present disclosure may be easily prepared by the standard procedure in the field. The genes encoding these proteins may then be obtained from the microorganisms.

Due to the redundancy of genetic codons, many different DNA sequences may encode the same amino acid sequence. The generation of these alternative DNA sequences encoding the same or substantially same protein is within the technological level of those skilled in the art. These various DNA sequences are included within a scope of the present disclosure. The “substantially same” sequence refers to a sequence with amino acid substitution, deletion, addition or insertion that does not substantially affect the insecticidal activity, and also includes a fragment that retains the insecticidal activity.

The substitution, deletion or addition of the amino acid sequence in the present disclosure is a routine technology in the field, preferably such an amino acid change is: a small property change, namely conservative amino acid substitution that does not significantly affect the folding and/or activity of the protein; small deletion, typically deletion of about 1-30 amino acids; small amino- or carboxy-terminal extension, for example, amino-terminal extension of one methionine residue; and a small linker peptide, for example, the length of about 20-25 residues.

Examples of the conservative substitution are those that occur within the following amino acid groups: basic amino acids (such as an arginine, a lysine, and a histidine), acidic amino acids (such as a glutamic acid and an aspartic acid), polar amino acids (such as a glutamine, and an asparagine), hydrophobic amino acids (such as a leucine, an isoleucine, and a valine), aromatic amino acids (such as a phenylalanine, a tryptophan, and a tyrosine), and small molecular amino acids (such as a glycine, an alanine, a serine, a threonine, and a methionine). Those amino acid substitutions that generally do not change the specific activity are well-known in the field, and already described, for example, by N. Neurath and R. L. Hill in “Protein” published by Academic Press, New York in 1979. The most common interchanges are Ala/Ser, Val/lle, Asp/Glu, Thu/Ser, Ala/Thr, Ser/Asn, Ala/Val, Ser/Gly, Tyr/Phe, Ala/Pro, Lys/Arg, Asp/Asn, Leu/lle, Leu/Val, Ala/Glu and Asp/Gly, and their opposite interchanges.

It is apparent to those skilled in the art that such substitutions may occur outside areas important to the function of the molecule, and still produce the active polypeptide. The amino acid residues that are essential for the activity of the polypeptide of the present disclosure and are therefore selected not to be substituted may be identified according to methods known in the field, such as site-directed mutagenesis or alanine-scanning mutagenesis (referring to, for example, Cunningham and Wells, 1989, Science 244: 1081-1085). The latter technology is to introduce a mutation at each positively charged residue in the molecule, and to test the inhibitory activity of the mutant molecules obtained, thereby the amino acid residues that are important to the activity of the molecule are determined. Substrate-enzyme interaction sites may also be determined by analysis of its three-dimensional structure, this three-dimensional structure may be determined by technologies such as nuclear magnetic resonance analysis, crystallography, or photoaffinity labeling (referring to, for example, de Vos et al., 1992, Science 255:306-312; Smith et al., 1992, J. Mol. Biol 224:899-904; and Wlodaver et al., 1992, FEBS Letters 309:59-64).

In the present disclosure, the ACh1 protein includes, but is not limited to, SEQ ID NO:1 or SEQ ID NO:2, and amino acid sequences having certain identity with the amino acid sequence shown in SEQ ID NO:1 or SEQ ID NO:2 are also included in the present disclosure. These sequences are typically greater than 78% of similarity/identity of the sequence of the present disclosure, preferably greater than 85%, more preferably greater than 90%, even more preferably greater than 95%, and may be greater than 99%. Preferred polynucleotides and proteins of the present disclosure may also be defined according to more specific ranges of the identity and/or similarity. For example, there are 78%, 79%, 80%, 81%, 82%, 83%, 84%, 85%, 86%, 87%, 88%, 89%, 90%, 91%, 92%, 93%, 94%, 95%, 96%, 97%, 98% or 99% of the identity and/or similarity with the sequences exemplified in the present disclosure.

The regulatory sequence described in the present disclosure includes, but is not limited to, a promoter, a transit peptide, a terminator, an enhancer, a leader sequence, an intron, and other regulatory sequences operably linked to the ACh1 protein.

The promoter is a promoter expressible in the plant, and the “promoter expressible in the plant” refers to a promoter that ensures the expression of the coding sequence linked to it in plant cells. The promoter expressible in the plant may be a constitutive promoter. Examples of the promoter that direct the constitutive expression in the plant include, but are not limited to, a 35S promoter derived from a cauliflower mosaic virus, a maize Ubi promoter, a promoter of a rice GOS2 gene and the like. Alternatively, the promoter expressible in the plant may be a tissue-specific promoter, namely the promoter directs the expression of the coding sequence to a higher level in some tissues of the plant, such as in a green tissue, than in other tissues of the plant (may be determined by a conventional RNA test), such as a PEP carboxylase promoter. Alternatively, the promoter expressible in the plant may be a wound-inducible promoter. The wound-inducible promoter or a promoter that directs a wound-induced expression pattern means that the expression of the coding sequence under the control of the promoter is significantly increased while the plant is subjected to a mechanical or insect-induced wound compared to normal growth conditions. Examples of the wound-inducible promoter include, but are not limited to, promoters of protease inhibitory genes (pin l and pin ll) of potato and tomato and a promoter of a maize protease inhibitor gene (MPI).

The transit peptide (also known as a secretion signal sequence or a targeting sequence) directs a transgenic product to a specific organelle or cellular compartment, the transit peptide may be heterologous to the receptor protein, for example, by using a transit peptide sequence encoding a chloroplast to target the chloroplast, or using a ‘KDEL’ retention sequence to target an endoplasmic reticulum, or using CTPP of a barley lectin gene to target a vacuole.

The leader sequence includes, but is not limited to, a picornavirus leader sequence, such as an encephalomyocarditis virus 5′ non-coding region (EMCV) leader sequence; a potato Y virus group leader sequence, such as a maize dwarf mosaic virus (MDMV) leader sequence; a human immunoglobulin heavy chain binding protein (BiP); an untranslated leader sequence of coat protein mRNA of alfalfa mosaic virus (AMV RNA4); and a tobacco mosaic virus (TMV) leader sequence.

The enhancer includes, but is not limited to, a cauliflower mosaic virus (CaMV) enhancer, a figwort mosaic virus (FMV) enhancer, a carnation etched ring virus (CERV) enhancer, a cassava vein mosaic virus (CsVMV) enhancer, a mirabilis mosaic virus (MMV) enhancer, a cestrum yellow leaf curling virus (CmYLCV) enhancer, a cotton leaf curl multan virus (CLCuMV), a commellna yellow motlle virus (CoYMV) and a peanut chlorella leaf strip virus (PCLSV) enhancer.

For monocot applications, the intron includes, but is not limited to, a maize hsp70 intron, a maize ubiquitin intron, an Adh intron 1, a sucrose synthase intron, or a rice Act1 intron. For dicot applications, the intron includes, but is not limited to, a CAT-1 intron, a pKANNIBAL intron, a PIV2 intron, and a “super ubiquitin” intron.

The terminator may be a suitable polyadenylation signal sequence functioning in the plant, including, but not limited to, a polyadenylation signal sequence derived from a nopaline synthase (NOS) gene of Agrobacterium tumefaciens, a polyadenylation signal sequence derived from a protease inhibitor ll (pin ll) gene, a polyadenylation signal sequence derived from a pea ssRUBISCO E9 gene, and a polyadenylation signal sequence derived from a α-tubulin gene.

The “operably linked” in the present disclosure refers to association of nucleic acid sequences such that one sequence may provide a desired function for the linked sequence. In the present disclosure, the “operably linked” may be to link a promoter with an interested sequence, so that the transcription of the interested sequence is controlled and regulated by the promoter. While the interested sequence encodes a protein and the expression of the protein is desired, the “operably linked” means that: the promoter is linked to the sequence, so that an obtained transcript is efficiently translated in a linkage mode. If the linkage of the promoter to the coding sequence is transcript fusion and the expression of the encoded protein is desired, such linkage is manufactured, so that the first translation initiation codon in the obtained transcript is an initiation codon of the coding sequence. Alternatively, if the linkage of the promoter to the coding sequence is translational fusion and the expression of the encoded protein is desired, such linkage is manufactured, so that the first translation initiation codon contained in a 5′ untranslated sequence is linked to the promoter, and a relationship between an obtained translation product and a translational open reading frame encoding the desired protein accords with the reading frame in the linkage mode. The nucleic acid sequence that may be “operably linked” includes, but is not limited to: sequences providing gene expression functions (namely gene expression elements such as a promoter, a 5′ untranslated region, an intron, a protein coding region, a 3′ untranslated region, a poly adenylation site and/or a transcription terminator), sequences providing DNA transfer and/or integration functions (namely a T-DNA border sequence, a site-specific recombinase recognition site, and an integrase recognition site), sequences providing selectivity functions (namely an antibiotic resistance marker, and a biosynthetic gene), sequences providing scoreable marker functions, sequences that assist in sequence operation in vitro or in vivo (namely a polylinker sequence, and a site-specific recombination sequence) and sequences providing replication functions (namely a bacterial replication origin, a autonomously replicating sequence, and a centromeric sequence).

In the present disclosure, the “insecticide” or “insect resistance” means that it is toxic to crop pests, thereby the “control” and/or “prevention” of the crop pests is achieved. Preferably, the “insecticide” or “insect resistance” means that the crop pests are killed. More specifically, the target insect is the a thrip pest.

The ACh1 protein in the present disclosure is virulent to the a thrip pest. The plant in the present disclosure, especially the corn, the soybean and the cotton, contains an exogenous DNA in its genome. The exogenous DNA contains a nucleotide sequence encoding the ACh1 protein. The a thrip pest is in contact with the protein by ingesting the plant tissue, and after the contact, the growth of the a thrip pest is inhibited and/or death is caused. The inhibition means lethal or sub-lethal. At the same time, the plant should be morphologically normal, and may be cultivated under a conventional method for consumption and/or generation of products. In addition, the plant may substantially eliminate the need for a chemical or biological pesticide (the chemical or biological pesticide is an insecticide against the a thrip pest targeted by the ACh1 protein).

The expression level of an insecticidal protein (a β- pore-forming protein) in the plant material may be detected by a plurality of methods described in the field, for example, by applying a specific primer to quantify mRNA encoding the insecticidal protein produced in the tissue, or directly specifically detecting the amount of the insecticidal protein produced.

Different tests may be applied to determine the insecticidal effect of the β- pore-forming protein in the plant. In the present disclosure, the target insect is mainly the a thrip pest.

In the present disclosure, the ACh1 protein may have an amino acid sequence shown in SEQ ID NO:1 or SEQ ID NO:2 in a sequence listing. In addition to the coding region containing the ACh1 protein, other elements may also be included, such as a protein encoding a selectable marker.

In addition, an expression cassette containing the polynucleotide sequence encoding the ACh1 protein of the present disclosure may also be expressed in the plant together with at least one protein encoding a herbicide resistance gene, the herbicide resistance gene includes, but not limited to, a glufosinate-ammonium resistance gene (such as a bar gene, and a pat gene), a Betanal resistance gene (such as a pmph gene), a glyphosate resistance gene (such as an EPSPS gene), a bromoxynil resistance gene, a sulfonylurea resistance gene, an anti-herbicide dalapon resistance gene, an anti-cyanamide resistance gene or a resistance gene of a glutamine synthase inhibitor (such as PPT), as to obtain the transgenic plant having both high insecticidal activity and herbicide resistance.

In the present disclosure, the exogenous DNA is introduced into the plant, for example, the gene or expression cassette or recombinant vector encoding the ACh1 protein is introduced into the plant cell, and the conventional transformation method includes, but not limited to, agrobacterium-mediated transformation, micro-emission bombardment, direct DNA ingestion into a protoplast, electroporation, or whisker silicon-mediated DNA introduction.

The present disclosure provides a use of an insecticidal protein and has the following advantages.

1. Prevention and treatment of internal causes: The prior arts mainly control the harm of the a thrip pest by the external action namely the external causes, for example, the agricultural control, the chemical control, the physical control and the biological control; and the present disclosure controls the a thrip pest by producing the ACh1 protein that may kill the a thrip pest in the plant, namely the a thrip pest is controlled by the internal causes.

2. No pollution and no residue: Although the chemical control method used in the prior art plays a certain role in controlling the harm of the a thrip pest, it also brings the pollution, damage and residue to humans, livestocks and farmland ecosystems; and using the method of the present disclosure to control the a thrip pest, the above adverse consequences may be eliminated.

3. Prevention and control during whole growth period: The methods used in the prior arts to control the a thrip pest are all by stages, and the present disclosure is to protect the plant during the whole growth period, and the transgenic plant (ACh1 protein) may be prevented from being attacked by the a thrip pest from germination, growth, to flowering and fruiting.

4. Whole plant control: Most of the methods used in the prior art to control the a thrip pest are localized, such as foliar spraying; and the present disclosure protects the entire plant, for example, roots, leaves, stems, fruits, tassels, female ears, anthers or filaments of the transgenic plant (ACh1 protein) are all resistant to the attack of the a thrip pest.

5. Stable effect: Whether it is the agricultural control method or the physical control method used in the prior art, it is necessary to use the environmental conditions to control the pests, and there are many variable factors; the present disclosure is to express the ACh1 protein in the plant, which effectively overcomes the disadvantages of the unstable environmental conditions, and the control effect of the transgenic plant (ACh1 protein) of the present disclosure is stable and consistent in different places, different times and different genetic backgrounds.

6. Simpleness, convenience and economy: The present disclosure only needs to plant the transgenic plant capable of expressing the ACh1 protein, and does not need to adopt other measures, thereby reducing a lot of manpower, material resources and financial resources.

7. Complete effect: The methods used in the prior art to control the a thrip pest are not thorough in effect, and only play a role in relieving; and the transgenic plant (ACh1 protein) of the present disclosure may cause a large number of deaths of the newly hatched larvae of the a thrip pest.

The technical schemes of the present disclosure are further described in detail below by drawings and embodiments.

### BRIEF DESCRIPTION OF THE DRAWINGS

FIG. 1 is a construction flowchart of a recombinant expression vector DBN01-T containing an ACh1 nucleotide sequence for the use of the insecticidal protein according to the present disclosure.

FIG. 2 is a construction flowchart of a recombinant expression vector DBN01-B containing an ACh1 nucleotide sequence for the use of the insecticidal protein according to the present disclosure.

### DETAILED DESCRIPTION OF THE EMBODIMENTS

The technical schemes of the use of the insecticidal protein of the present disclosure are further described below by specific embodiments.

### First Embodiment: Acquisition and Synthesis of Gene

### 1. Acquisition of the Nucleotide Sequence

An amino acid sequence of an ACh1_1 insecticidal protein (309 amino acids) is shown in SEQ ID NO:1 in a sequence listing. An ACh1_1 nucleotide sequence (930 nucleotides) encoding the amino acid sequence corresponding to the ACh1_1 insecticidal protein is shown in SEQ ID NO: 3 in the sequence listing.

An amino acid sequence of an ACh1_4 insecticidal protein (309 amino acids) is shown in SEQ ID NO:2 in the sequence listing. An ACh1_4 nucleotide sequence (930 nucleotides) encoding the amino acid sequence corresponding to the ACh1_4 insecticidal protein in bacteria is shown in SEQ ID NO: 4 in the sequence listing.

### 2. Synthesis of Above Nucleotide Sequence

The nucleotide sequences (as shown in SEQ ID NO:3 or SEQ ID NO:4 in the sequence listing) of ACh1_1 and ACh1_4 are synthesized by Nanjing GenScript Biotech Corp.

### Second Embodiment: Construction Of Recombinant Expression Vector And Transformation Of Recombinant Expression Vector Into Agrobacterium Tumefaciens To Obtain Ach1 Protein

### 1. Construction of Recombinant Cloning Vector Containing ACh1 Gene

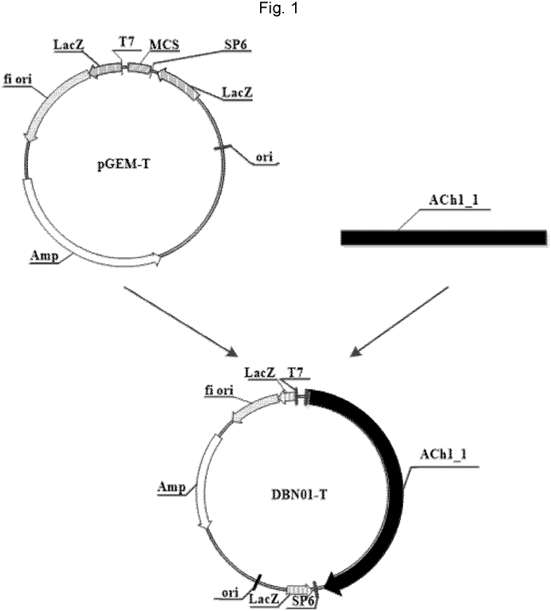

The synthesized ACh1_1 nucleotide sequence is linked to a cloning vector pGEM-T (Promega, Madison, USA, CAT: A3600), and an operation step is performed according to instructions of a pGEM-T vector product of Promega Company, to obtain a recombinant cloning vector DBN01-T, and its construction process is shown in FIG. 1 (herein, Amp represents an ampicillin resistance gene; f1 represents the origin of replication of phage f1; LacZ is an LacZ initiation codon; SP6 is an SP6 RNA polymerase promoter; T7 is a T7 RNA polymerase promoter; ACh1_1 is the ACh1_1 nucleotide sequence (SEQ ID NO:3); and MCS represents multiple cloning sites).

Then, the recombinant cloning vector DBN01-T is transformed into Escherichia coli T1 competent cells (Transgen, Beijing, China, CAT: CD501) by a heat shock method, and a white bacterial colony is picked, and placed in a Luria-Bertani (LB) liquid medium (10 g/L of a tryptone, 5 g/L of a yeast extract, 10 g/L of NaCl, 100 mg/L of an ampicillin, and pH is adjusted to 7.5 with NaOH) and cultured overnight at 37° C. Plasmids thereof are extracted by an alkaline method and stored at -20° C. for future use.

After the extracted plasmid is identified by enzyme digestion, the positive colonies are sequenced and verified, and results show that the ACh1_1 nucleotide sequence inserted in the recombinant cloning vector DBN01-T is the nucleotide sequence shown in the sequence listing (SEQ ID NO:3). That is to say, the ACh1_1 nucleotide sequence is correctly inserted.

According to the aforementioned method of constructing the recombinant cloning vector DBN01-T, the synthesized ACh1_4 nucleotide sequence was ligated into a cloning vector pGEM-T to obtain a recombinant cloning vector DBN02-T, wherein ACh1_4 represented the ACh1_4 nucleotide sequence (SEQ ID NO: 4). The correct insertion of the ACh1_4 nucleotide sequence in the recombinant cloning vector DBN02-T was verified through enzyme digestion and verification by sequencing.

### 2. Construction of the Recombinant Expression Vector Containing the ACh1 Gene

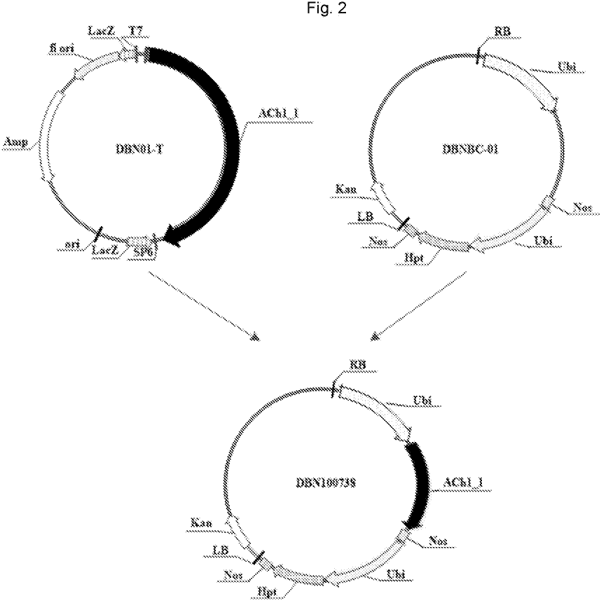

The recombinant cloning vector DBN01-T and the expression vector DBNBC-01 (vector framework: pCAMBIA2301 (provided by the CAMBIA institution)) are digested with restriction endonucleases, and an excised ACh1_1 nucleotide sequence fragment is inserted between the restriction endonuclease sites of the expression vector DBNBC-01. It is well-known to those skilled in the art to construct a vector with a conventional enzyme digestion method, the recombinant expression vector DBN01-B is constructed, and the construction flow is shown in FIG. 2 (Kan: kanamycin gene; RB: right border; prUbi: maize ubiquitin gene promoter (SEQ ID NO:5); ACh1_1: ACh1_1 plant nucleotide sequence ( SEQ ID NO:3); tNos: terminator of nopaline synthase gene (SEQ ID NO:6); Hpt: hygromycin phosphotransferase gene (SEQ ID NO:7); and LB: left border).

The recombinant expression vector DBN01-B is transformed into the Escherichia coli T1 competent cells with the heat shock method; the white colony is picked and placed in the LB liquid medium (10 g/L of the tryptone, 5 g/L of the yeast extract, 10 g/L of NaCl, 50 mg/L of the kanamycin, and pH is adjusted to 7.5 with NaOH); and culture is performed overnight at 37° C., and plasmids thereof are extracted by an alkaline method. The extracted plasmid is identified by the restriction endonuclease digestion, and the positive colonies are sequenced and identified. The results show that the nucleotide sequence in the recombinant expression vector DBN01-B is the nucleotide sequence shown in SEQ ID NO:3 in the sequence listing, that is, the ACh1_1 nucleotide sequence.

According to the aforementioned method of constructing the recombinant expression vector DBN01-B, the ACh1_4 nucleotide sequence cleaved from the recombinant cloning vector DBN02-T by enzymatic cleaving was inserted into the expression vector DBNBC-01 to obtain a recombinant expression vector DBN02-B. After enzymatic cleaving and as verified by sequencing, it is found that the nucleotide sequence in the recombinant expression vector DBN02-B contained the nucleotide sequence as shown in SEQ ID NO: 4 of the sequence listing, namely the ACh1_4 nucleotide sequence. The ACh1_4 nucleotide sequence could be connected to the Ubi promoter and the Nos terminator.

### 3. Transformation of the Recombinant Expression Vector Into an Agrobacterium

The correctly constructed recombinant expression vectors DBN01-B and DBN02-B are transformed into agrobacterium LBA4404 (lnvitrgen, Chicago, USA, CAT: 18313-015) through a liquid nitrogen method, and the transformation conditions are as follows: 100 µl of the agrobacterium LBA4404, and 3 µl of a plasmid DNA (the recombinant expression vector); it is placed in liquid nitrogen for 10 minutes, and a warm water bath is performed at 37° C. for 10 minutes; the transformed agrobacterium LBA4404 is inoculated in an LB tube, cultured for 2 hours under conditions of a temperature of 28° C. and a rotation speed of 200 rpm, and spread on an LB plate containing 50 mg/L of rifampicin and 100 mg/L of kanamycin until positive monoclones grow, the monoclones are picked for culture and plasmids thereof are extracted, the restriction endonuclease is used to verify the recombinant expression vectors DBN01-B and DBN02-B after being enzyme-digested, and results show that the structure of the recombinant expression vectors DBN01-B and DBN02-B is completely correct.

### Example 3. Obtaining of Transgenic Corn Plant

According to the conventional agrobacterium infection method, the immature embryos of the aseptically cultured maize variety Zong 31 (Z31) are co-cultured with the agrobacterium transformed with the recombinant expression vector described in step 3 in the second embodiment, to transfer the T-DNA (including the promoter sequence of maize ubiquitin gene, the ACh1_1 nucleotide sequence, the ACh1_4 nucleotide sequence, the Hpt gene and the Nos terminator sequence) in the recombinant expression vectors DBN01-B and DBN02-B constructed in step 2 in the second embodiment into a maize genome, so as to obtain a corn plant transformed with the ACh1_1 nucleotide sequence and a corn plant transformed with the ACh1_4 nucleotide sequence. In addition, a wild corn plant is used as a control.

For agrobacterium-mediated transformation of corns, briefly, immature embryos are isolated from the corns and are in contact with agrobacterium suspension. The agrobacterium can deliver the ACh1_1 nucleotide sequence and/or the ACh1_4 nucleotide sequence to at least one cell (step 1: infection step) of one of the embryos. In this step, the embryos are preferably immersed in the agrobacterium suspension (OD₆₆₀=0.4-0.6, an infection medium (4.3 g/L of MS salt, MS vitamins, 300 mg/L of casein, 68.5 g/L of sucrose, 36 g/L of glucose, 40 mg/L of Acetosyringone (AS), and 1 mg/L of 2,4-dichlorophenoxyacetic acid (2,4-D), pH 5.3) to initiate inoculation. The embryos are co-cultured with the agrobacterium for a period of time (3 days) (Step 2: co-culture step). Preferably, the embryos are cultured in a solid culture medium (4.3 g/L of the MS salt, the MS vitamins, 300 mg/L of casein, 20 g/L of sucrose, 10 g/L of glucose, 100 mg/L of AS, 1 mg/L of 2,4-D, and 8 gIL of agar, pH5.8) after the infection step. After this co-culture phase, there may be an optional “recovery” step. In the “recovery” step, in a recovery culture medium (4.3 g/L of the MS salt, the MS vitamins, 300 mg/L of casein, 30 g/L of sucrose, 1 mg/L of 2,4-D, and 8 g/L of agar, pH5.8), there is at least one antibiotic (cephalosporin) known to inhibit the growth of the agrobacterium, and a selective agent for a plant transformant (Step 3: recovery step) is not added. Preferably, the embryos are cultured on a solid medium with the antibiotic without the selective agent, as to eliminate the agrobacterium and provide a recovery period for infected cells. Next, the inoculated embryos are grown on a culture medium containing the selective agent (hygromycin) and a grown transformed callus is selected (Step 4: selection step). Preferably, the embryos are cultured in the solid culture medium (4.3 g/L of the MS salt, the MS vitamins, 300 mg/L of casein, 5 g/L of sucrose, 50 mg/L of hygromycin, 1 mg/L of 2,4-D, and 8 g/L of agar, pH5.8) containing the selective agent, so as to cause the transformed cells to selectively grow. The callus are then regenerated into plants (Step 5: regeneration step), preferably, the callus grown on the medium containing the selective agent is cultured on the solid medium (MS differentiation medium and MS rooting medium) to regenerate the plant.

The screened resistant callus are transferred to the MS differentiation medium (4.3 g/L of the MS salt, the MS vitamins, 300 mg/L of casein, 30 g/L of sucrose, 2 mg/L of 6-benzyladenine, 50 mg/L of hygromycin, and 8 g/L of agar, pH5.8), and culture differentiation is performed at 25° C. The differentiated seedling is transferred to the MS rooting medium (2.15 g/L of the MS salt, the MS vitamins, 300 mg/L of casein, 30 g/L of sucrose, 1 mg/L of indole-3-acetic acid, and 8 g/L of agar, pH5.8); and the seedling is cultured to a height of about 10 cm at 25° C., and then moved to a greenhouse to grow until fruiting. In the greenhouse, culture is performed for 16h at 28° C. every day, and then culture is performed for 8h at 20° C.

### Example 4. Verification of Transgenic Corn Plants With TaqMan

About 100 mg of leaves of the corn plant transformed with the ACh1_1 nucleotide sequence or the ACh1_4 nucleotide sequence is taken as a sample, respectively, and the genome DNA is extracted with DNeasy Plant Maxi Kit of Qiagen, and the copy number of the Hpt gene is detected by a Taqman probe fluorescence quantitative PCR method to determine the copy number of the ACh1_1 gene or the ACh1_4 gene. At the same time, the wild corn plant is used as a control, and the detection and analysis are performed according to the above method. The experiment is repeated for 3 times, and the average value is taken.

A specific method to detect the copy number of the Hpt gene is as follows.

Step 11, 100 mg of the leaves of the corn plant transformed with the ACh1_1 nucleotide sequence, the ACh1_4 nucleotide sequence and the wild corn plant are taken respectively, and ground into uniform slurry with liquid nitrogen in a mortar, and 3 replicates for each sample are taken.

Step 12, Qiagen’s DNeasy Plant Mini Kit is used to extract the genome DNA of the above samples, and a specific method refers to its product specification.

Step 13, NanoDrop 2000 (Thermo Scientific) is used to measure the genome DNA concentration of the above samples.

Step 14, the genome DNA concentration of the above samples is adjusted to the same concentration value, and the range of the concentration value is 80-100 ng/µl.

Step 15, the Taqman probe fluorescence quantitative PCR method is used to identify the copy number of the sample, the sample with the known copy number after the identification is used as a standard substance, and the sample of the wild corn plant is used as a control, 3 replicates for each sample are taken, and its average value is taken; and fluorescence quantitative PCR primer and probe sequences are as follows.

The following primers and probes are used to detect the Hpt nucleotide sequence.

Primer 1: cagggtgtcacgttgcaaga is as shown in SEQ ID NO:8 in the sequence listing.

Primer 2: ccgctcgtctggctaagatc is as shown in SEQ ID NO:9 in the sequence listing.

Probe 1: tgcctgaaaccgaactgcccgctg is as shown in SEQ ID NO: 10 in the sequence listing.

A PCR reaction system is as follows.

| | |
|---|---|
| JumpStart™ Taq ReadyMix™ (Sigma) | 10 µl |
| 50× primer/probe mixture | 1 µl |
| Genomic DNA | 3 µl |
| Water (ddH₂O) | 6 µl |

The 50× primer/probe mixture contains 45 µl of each primer at a concentration of 1 mM, 50 µl of the probe at a concentration of 100 µM and 860 µl of 1×TE buffer, and is stored in a centrifuge tube at 4° C.

PCR reaction conditions are as follows.

| Step | Temperature | Time |
|---|---|---|
| 21 | 95° C. | 5 min |
| 22 | 95° C. | 30 s |
| 23 | 60° C. | 1 min |
| 24 | Returning to Step 22, and repeating for 40 times | |

Data is analyzed with SDS 2.3 software (Applied Biosystems).

The experimental results by analyzing the copy number of the Hpt genes show that, the ACh1_1 nucleotide sequence and the ACh1_4 nucleotide sequence have been integrated into the genome of the tested corn plants, and the corn plants transformed with the ACh1_1 nucleotide sequence and the corn plants transformed with the ACh1_4 nucleotide sequence are all obtained with single-copy.

### Example 5. Detection of Insect Resistance of Transgenic Corn Plants

The corn plant transformed with the ACh1_1 nucleotide sequence, the corn plant transformed with the ACh1_1 nucleotide sequence, the corresponding wild-type corn plant, and the non-transgenic corn plant identified by Taqman are detected for insect-resistant effects against the Frankliniella occidentalis (Pergande).

Fresh leaves (heart leaves) of the corn plant transformed with the ACh1_1 nucleotide sequence, of the corn plant transformed with the ACh1_4 nucleotide sequence, of the wild corn plant, and of the corn plant (Stage V3-V4) identified as non-transgenic by Taqman are taken respectively, washed with sterile water and dried with gauze; then, the veins are removed from the corn leaves, the leaves are cut into strips of about 1 cm × 4 cm, and 1 piece of the cut strip-like leaf is taken and put the leaf on a moisturizing filter paper at the bottom of a circular plastic petri dish; 10 Frankliniella occidentalis (Pergande) (larvae) are put in each petri dish; after the insect-testing petri dish is covered, the petri dish is put for 1 day under the conditions of a temperature of 26±1° C., a relative humidity of 70%-80%, and a photoperiod (light/dark) of 16:8 for 5 days. Then the mortality rate of the larvae of frankliniella occidentalis (Pergande) and leaf damage are counted. The mortality rate = the number of dead insects/total number of infected insects × 100%. A total of 3 lines (S1, S2 and S3) are transformed into ACh1_1 nucleotide sequence, 3 lines (S4, S5 and S6) are transformed into ACh1_4 nucleotide sequence, 1 line is identified as non-transgenic (NGM) by Taqman, and 1 line is identified as wild (CK). 5 plants are selected from each line for test, and each plant is tested repeatedly for 3 times. Results are shown in Table 1.

TABLE 1

Insect resistance experimental results of transgenic corn plants inoculated with Frankliniella occidentalis (Pergande)

| Serial number of proteins | Test insect |
|---|---|
| | Frankliniella occidentalis (Pergande) |
| ACh1_1 | + |
| ACh1_4 | + |
| NGM | - |
| CK | - |

“+” means that there is an inhibitory activity against pest; and “-” means that there is no inhibitory activity against pest

The results of Table 1 show that the corn plants transformed with the ACh1_1 nucleotide sequence, the corn plants transformed with the ACh1_4 nucleotide sequence both have had a good insecticidal effect against the Frankliniella occidentalis (Pergande), while the WT corn plants and the non-transgenic plants identified by Taqman are basically not lethal to larvae of Frankliniella occidentalis (Pergande).

The detection results also show that the corn plants transformed with the ACh1_1 nucleotide sequence and the corn plants transformed with the ACh1_4 nucleotide sequence are only slightly damaged.

Therefore, it indicates that the ACh1_1 protein and the ACh1_4 protein show resistance activity against the a thrip pest, and this activity is sufficient to have adverse effects on the growth of the a thrip pest, so that the a thrip pest can be controlled in the fields. In addition, it is also possible to reduce the occurrence of diseases on corns by controlling the damage of the a thrip pest, thereby greatly improving the yield and quality of the transgenic ACh1 plants.

In conclusion, through the use of the insecticidal protein of the present disclosure, ACh1 protein that can kill the a thrip pest is produced in a plant in vivo to control the a thrip pest. Compared with an agricultural control method, a chemical control method, a physical control method and a biological control method used in the prior art, the present disclosure achieves the protection of whole growth period and whole plant on the plants so as to control the infestation of the a thrip pest, and is pollution-free, residue-free, stable in effect, thorough, simple, convenient and economical.

Finally, it should be noted that the above embodiments are only used to illustrate the technical schemes of the present disclosure and not to limit them. Although the present disclosure is described in detail with reference to the preferred embodiments, those of ordinary skill in the art should understand that the technical schemes of the present disclosure may be modified or equivalently replaced without departing from the spirit and scope of the technical schemes of the present disclosure.

What is claimed is:

1. A method for controlling a thrip pest, comprising allowing the a thrip pest to be at least in contact with an ACh1 protein;
preferably, the ACh1 protein is present in a host cell that produces at least the ACh1 protein, and the a thrip pest is in contact with at least the ACh1 protein by ingesting the host cell; and
more preferably, the ACh1 protein is present in at least a bacterium or a transgenic plant that generates the ACh1 protein, the a thrip pest is in contact with at least the ACh1 protein by ingesting the bacterium or a tissue of the transgenic plant, and after contacting, the growth of the a thrip pest is inhibited and/or death is caused, so as to achieve the control of the damage of the a thrip pest to plants.

2. The method for controlling a thrip pest according to claim 1, wherein the transgenic plant is corn, soybean, cotton or rape.

3. The method for controlling a thrip pest according to claim 1, wherein the tissue of the transgenic plant is a leaf, a stem, a fruit, a male ear, a female ear, an anther, or a filament.

4. The method for controlling a thrip pest according to claim 1, wherein the ACh1 protein is an ACh1_1 protein or an ACh1_4 protein.

5. The method for controlling a thrip pest according to claim 4, wherein the ACh1 protein has an amino acid sequence shown in SEQ ID NO:1 or SEQ ID NO:2.

6. The method for controlling a thrip pest according to claim 4, wherein a nucleotide sequence of the ACh1 protein is shown in SEQ ID NO:3 or SEQ ID NO:4.

7. The method for controlling a thrip pest according to claim 1, wherein the transgenic plant further comprises at least a second nucleotide different from the nucleotide encoding the ACh1 protein.

8. The method for controlling a thrip pest according to claim 7, wherein the second nucleotide encodes a Cry-like insecticidal protein, a Vip-like insecticidal protein, a protease inhibitor, lectin, α-amylase, or a peroxidase.

9. The method for controlling a thrip pest according to claim 7, wherein the second nucleotide is a dsRNA that inhibits an important gene in a target insect pest.

10. The method for controlling a thrip pest according to claim 1, wherein the thrip pest is selected from the group consisting of Anaphothrips obscurus, Frankliniella tenuicornis (Uzel), Stenchaetothrips biformis (Bagnall) and frankliniella occidentalis (Pergande).

11. A method of producing a plant for controlling a thrip pest, comprising introducing a polynucleotide sequence encoding an ACh1 protein into a genome of the plant.

12. The method of producing a plant for controlling a thrip pest according to claim 11, wherein the polynucleotide sequence of the ACh1 protein is shown in SEQ ID NO:3 or SEQ ID NO:4.

13. The method of producing a plant for controlling a thrip pest according to claim 11, wherein the ACh1 protein has an amino acid sequence shown in SEQ ID NO:1 or SEQ ID NO:2.

14. A method of producing a plant seed for controlling a thrip pest, comprising hybridizing a plant obtained by the method according to claim 11 with a second plant, so as to produce a seed containing a polynucleotide sequence encoding an ACh1 protein.

15. A method of cultivating a plant for controlling a thrip pest, comprising
planting at least one plant seed, wherein the genome of the plant seed comprises a polynucleotide sequence encoding an ACh1 protein;
growing the plant seed into a plant; and
growing the plant under conditions that the a thrip pest is artificially inoculated and/or the hazard of the a thrip pest naturally occurs, and harvesting a plant that has an attenuated plant damage and/or has an increased plant yield compared with other plants that do not have the polynucleotide sequences encoding the ACh1 protein.

16. The method of cultivating a plant for controlling a thrip pest according to claim 15, wherein the polynucleotide sequence of the ACh1 protein is shown in SEQ ID NO:3 or SEQ ID NO:4.

17. The method of cultivating a plant for controlling a thrip pest according to claim 15, wherein the ACh1 protein has an amino acid sequence shown in SEQ ID NO:1 or SEQ ID NO:2.
